(19) United States
(12) Patent Application Publication
(10) Pub. No.: US 2004/0204497 A1
(43) Pub. Date: Oct. 14, 2004

(54) Metabolic enhancing properties of octopamine salts

(75) Inventors: Derek Wayne Cornelius, Cape Girardeau, MO (US); Gray Lee Haynes, Scott City, MO (US)

Correspondence Address: CREATIVE COMPOUNDS, LLC, P.O. BOX 4011, SCOTT CITY, MO, 63780, US

(73) Assignee: Creative Compounds, LLC

(21) Appl. No.: 10/408,797

(22) Filed: Apr. 8, 2003

(51) Int. Cl. A61K 31/137

(52) U.S. Cl. 514/649

(57) ABSTRACT

Octopamine salts are disclosed which have powerful metabolic enhancing effects in humans via oral administration. These salts not only have increased stability but also greatly increased bioavailability over the base form. The metabolic enhancing properties include positive blood sugar regulation, increased thermogenesis and fat loss, increased anticatabolism/anabolism, decreased appetite, improved mental focus and psychological outlook and increased energy production and anti-fatigue actions.

### REFERENCES CITED

U.S. Patent Documents

| | | |
|---|---|---|
| 6,340,482 | January, 2002 | Jones |
| 6,340,481 | January, 2002 | Jones |
| 6,316,499 | November, 2001 | Jones |
| 6,224,873 | May, 2001 | Jones |

### OTHER REFERENCES

Robertson H A, et al. “Octopamine in the insect central nervous system: distribution, biosynthesis and possible physiological role.” J Physiol. 237(2):34P-35P, 1974.

Juorio A V, et al. “The normal occurrence of octopmine in neural tissue of the Octopus and other cephalopods.” J. Neurochem. 22(2):271-80, 1974.

Boulton A A, et at. “Biosynthesis of cerebral phenolic amines. In vivo formation of p-tyramine, octopamine, and synephrine.” Can. J. Biochem. 50(3):261-7, 1972.

Wheaton T A, et al. “The distribution of tyramine, N-methyltyramine, hordenine, octopamine, and synephrine in higher plants.” Lloydia 33(2):244-54, 1970.

Yen S T, et al. “Stimulatory effect of octopamine on beta 3 adrenorecptors to lower the uptake of [14C]-deoxy-D-glucose into rat adipocytes in vitro.” J. Auton. Pharmacol. 18(1):13-9, 1998.

Galitzky J. et at. “Specific stimulation of adipose tissue adrenergic beta 3 receptors by octopamine.” C. R. Acad. Sci. III 316(5):519-23, 1993.

Fontana E. et al. “Effects of octopamine on lipolysis, glucose transport and amine oxidation in mammalian fat cells. Comp. Biochem. Physiol. C. Toxicol. Pharmacol. 125(i):3344.

Visentin V. et al. “Dual action of octopamine on glucose transport into adipocytes: inhibition via beta3-adrenoceptor activation and stimulation via oxidation by amine oxidases.” J. Pharmacol. Exp. Ther. 299(1):96-104, 2001.

Carpene C. et al. “Beta 3-adrenergic receptors are responsible for the adrenergic inhibition of insulin-stimulated glucose transport in rat adipocytes.” Biochem J. 296(Pt 1):99-105, 1993.

Carpene C. et al. “Selective activation of beta3-adrenoceptors by octopamine: comparative studies in mammalian fat cells.” Naunyn Schmiedebergs Arch. Pharmacol. 359(4):310-21, 1999.

### BACKGROUND OF THE INVENTION

Octopamine is a naturally occurring amine that is widely dispersed throughout both the plant and animal kingdom. In plants, it is most notably found in several citrus varieties such as Citrus aurantium also known as bitter orange. This amine is also found in invertebrates such ass octopi and insects in relatively large concentrations and acts on specific octopamine receptors. In vertebrates, octopamine occurs in minute concentrations in various organs such as the brain. It is of interest that octopamine has been shown to be the only naturally occurring beta3 adrenergic agonist with considerable activity towards the beta3 receptor. While other amines such as ephedrine and norephedrine indirectly stimulate all of the adrenergic receptors such as alpha1, alpha2, beta1, beta2 and beta3, octopamine is exclusively a beta3 receptor agonist. It is theorized that many of the metabolic enhancing properties of octopamine are resultant to the stimulation of the beta3 receptors.

It bas been previously disclosed that extracts of Citrus aurantium contain a series of biogenic amines which includes synephrine, tyramine, octopamine, hordenine and methyltyramine. Furthermore, U.S. Pat. Nos. 6,340,482, 6,340,481, 6,316,499, 6,224,873 disclose that extracts which contain any one or more of these biogenic amines can effectively be used for such things as weight loss, appetite regulation, athletic function regulation, and increasing muscle mass. Unfortunately, there are several drawbacks to this technology. First although all of the above mentioned amines naturally occur in Citrus aurantium, the only one that occurs in practically large enough quantities for commercial utilization is synephrine. It is for this reason that all commercially available Citrus Aurantium extracts are standardized solely for synephrine and not for any of the other amines. Octopamine naturally occurs in Citrus aurantium at a lower concentration than any of the other amines. It is for this reason that octopamine from Citrus aurantium cannot be commercially extracted and used for metabolic enhancing properties.

Even if octopamine could be extracted from Citrus aurantium in significant enough quantities to be potentially commercially viable, we have found that the base form of octopamine which occurs naturally in Citrus aurantium to have several major drawbacks. First, we have discovered that octopamine in the base form is very unstable and oxidizes rapidly when readily exposed to the atmosphere. In-house results show that the base form of octopamine degrades in a linear fashion at least for the first 6 months which has been found to be the half life of octopamine base in exposed conditions to the atmosphere.

Finally, we have discovered that the base form of octopamine has significant bioavailability problems. We have found that the minimum effective dosage of octopamine base is nearly four times that of this present invention. There is thus great need for an improvement to this technology which allows octopamine to be a commercially viable option for metabolic enhancement.

It is now disclosed that synthetic octopamine salts overcome the above mentioned problems. We have conclusively shown that octopamine salts can be commercially synthesized in an efficient manner so that they can be sold as a viable option for metabolic enhancement. Since octopamine must be administered in fairly high dosages to enhance human metabolism, it is not practical nor is it cost effective to extract the extremely small quantities that occur in Citrus aurantium. Synthetic octopamine salts in no way depend on Citrus aurantium or the octopamine that is contained therein, and can be manufactured in metric ton quantities at an efficiency which easily allows efficacious dosages of octopamine to be taken for metabolic enhancement.

It is also now disclosed that the octopamine salts of this invention do not suffer from the crippling problem, of being unstable in normal atmospheric conditions. While octopamine base which occurs in Citrus aurantium oxidizes and degrades readily in the presence of oxygen, octopamine salts of this invention do not have this problem at all or it is significantly reduced so as to make it of little concern. For instance, although the half-life of octopamine base is roughly 6 months, the half-life for octopamine salts of this invention such as octopamine HCl is at least 5 years according to our calculations. To be used commercially for metabolic enhancement, it is of extreme importance that the octopamine form used has a shelf life long enough so that the octopamine remains filly efficacious for at least one year and preferably two years.

It is finally disclosed that the octopamine salts of this invention are much more bioavailable and therefore metabolically more potent than the corresponding octopamine base. We have found the octopamine salts of this invention are up to four times more efficient at affecting specific metabolic parameters than octopamine base.

The octopamine salts of this invention can be commercially used in various ways as metabolic enhancers. For instance, we have found usefulness with these compounds as weight loss agents. Unlike other, previous weight loss technology these octopamine salts cause little or no central nervous system stimulation and do not make the user feel “wired”, jittery, or anxious. Even in high dosages, the user feels no noticeable negative effect on his general feeling or overall state of well-being. Furthermore, these octopamine salts specifically enhance the loss of adipose tissue while preserving lean body mass such as muscle tissue. A significant problem for anyone trying to lose unwanted adipose tissue is the concurrent loss of lean body mass. Lean body mass includes tissues such as muscle, kidney, liver, heart and bone. For a person to feel healthy and strong, these tissues must also be healthy and strong. If, while dieting or taking a weight reducing agent, lean body mass is decreased, it has an immediate affect on the overall health and well-being of the organism. The octopamine salts of this invention preserve the precious lean body mass at the expense of adipose tissue. The effect is what is known as nutrient partitioning where nutrients are preserved or partitioned into lean body mass and expended or metabolically burned in adipose tissue.

It is also disclosed that the octopamine salts of this invention can be used as weight reducing agents either with or without a calorie reducing diet or exercise. Although any weight reducing program works better when calories are restricted and aerobic exercise is performed, we have found that this is not necessary. These octopamine salts cause a modest decrease in fat mass even with making no changes to a users' current diet and exercise regimen.

It is also disclosed, that the octopamine salts of this invention increase a user's overall metabolic rate. Thus, although a specific adipose reducing effect is achieved from the nutrient partitioning effect, these octopamine salts also actually cause an increase in the amount of calories that are actually burned or expended. By increasing the overall metabolic rate, fatigue is significantly reduced and perceived energy and ability to accomplish vigorous work related tasks is amplified.

It is also disclosed that the octopamine salts of this invention reduce the appetite for food of the user. Although this effect is weak, it nevertheless contributes to the overall effectiveness of the invention.

A further disclosure is that the octopamine salts of this invention have a sustained and noticeable effect on a person's focus and acuity as well as a person's psychological outlook. Subjective feedback confirms that the octopamine salts of this invention have very definite anti-depression functions and gives the user a positive attitude about life. Furthermore, feedback suggests that octopamine increases the ability of the user to focus and concentrate on difficult tasks such as test taking and problems solving.

Finally, it is disclosed that the octopamine salts of this invention are potent agents in regulating blood sugar and insulin levels. We have found that they specifically increase insulin sensitivity in muscle tissue apart from adipose tissue. This may help explain the nutrient partitioning effects of this invention. Insulin is an anabolic hormone to all tissues. Thus, increasing the ratio of the effectiveness of insulin in muscle as compared to adipose tissue would practically help a user preserve or increase muscle mass while decreasing fat mass.

We have found conclusively that a wide range of organic and inorganic octopamine salts conform to the scope of this present invention. The range of salts which conforms to this invention includes but is not limited to inorganic salts such as the hydrochloride, hydrobromide, sulfate, phosphate, nitrate, carbonate, hydroselenate, metasilicate, permanganate, etc. Organic salts likewise have been shown to conform to the scope of this invention and include but are not limited to carboxylic acids such as citrate malate, formate, tartrate, maleate, fumarate, rate, picolinate, nicotinate, pyruvate, succinate, glycinate, glutamate, ascorbate, decanoate, palmitate, oleate, acetate, propionate, etc. In respect to di-, tri- and tetr-carboxylic acids, the scope of this invention includes dioctopamine, trioctopamine and tetraoctopamine carboxylic salts. Finally, other organic salts such as organic sulfonic, carbonic, and phosphonic, salts are included in the scope of this patent. Examples of these are methanesulfonate, isethionate, toluenesulfonate, ethylcarbonate, metlylicarbonate, etc.

Human dosaging for the octopamine salts of this invention is most preferable in the range of 5-15 mg/kg of bodyweight calculated as base octopamine. The inventors have found that even at very high dosaging above 15 mg/kg, the octopamine salts of this invention are well tolerated. Daily dosaging below 5 mg/kg may also be useful when this invention is combined with other agents known in the art to cause weightloss such as yohimbine, ephedrine/ephedra, synephrine, green tea extract, etc. Although the octopamine salts of this invention can be dosed once per day for a desired effect, we have found that it is best to divide the dosage into two or three equal amounts given eight to twelve hours apart. This helps to ensure steady blood values and an overall more powerful, consistent effect. Effective dosaging may be in the form of tablets, capsule, sachets, effervescent powder or tablets, softgels, liquid or other oral delivery system known in the art.

### EXAMPLE 1

Octopamine base, octopamine HCl, octopamine malate, and octopamine ethylcarbonate were prepared and given as capsules in divided dosages at 6 mg/kg per day calculated as octopamine base. Subjects did not change their diet or exercise regimen. The following average results were achieved in terms of weight loss.

| | Week 1 | Week 4 | Week 8 |
|---|---|---|---|
| Octopamine base | 0 | 1 | 2 |
| Octopamine HCl | 2 | 6 | 9 |
| Octopamine malate | 1 | 5 | 10 |
| Octopamine ethylcarbonate | 2 | 6 | 10 |
| Control | 0 | 1 | 0 |

As can be seen, there was significant weight loss that was achieved by the octopamine salts of this invention. However, neither the octopamine base nor the control resulted in appreciable or significant weight loss. This attests to the outstandingly higher biopotency of the octopamine salts of this invention.

### EXAMPLE 2

Octopamine HCl was prepared and given as capsules in divided dosages at 6 mg/kg per day calculated as octopamine base. Subjects did not change their diet or exercise regimen. The following average results were achieved in terms of change in percent bodyfat and change in percent lean body mass. Subjective results are also shown regarding perceived energy and mental outlook and focus (scale of 1-10):

| | Week 1 | Week 4 | Week 8 |
|---|---|---|---|
| Change Percent Bodyfat | −.5% | −2% | −4% |
| Change Percent Lean Body Mass | 0% | +.3% | +1% |
| Perceived Energy | 6 | 8 | 8 |
| Perceived Mental Outlook and Focus | 7 | 7 | 9 |

These results clearly indicate the nutrient partitioning effect of the octopamine salts of this invention. Without changing diet or exercise, there was an average decrease in bodyfat with a simultaneous increase in lean body mass. Furthermore it seen that a statistically significant increase in perceived energy as well as perceived mental outlook and focus was achieved.

1. Bioavailable and environmentally stable octopamine salts which have powerful metabolic enhancing properties in humans via oral administration.

2. Metabolic enhancing properties according to claim 1 which include blood sugar regulation.

3. Blood sugar regulation according to claim 2 which includes increased insulin sensitivity and lower mean blood sugar values.

4. Metabolic enhancing properties according to claim 1 which include decreased appetite.

5. Metabolic enhancing properties according to claim 1 which include increased thermogenesis and weight loss.

6. Weight loss according to claim 5 which is predominantly from adipose tissue and not from lean body mass.

7. Weight loss according to claim 5 which is not dependent on changing a person's diet or exercise regimen.

8. Metabolic enhancing properties according to claim 1 which include increased anabolism/anticatabolism of lean body mass.

9. Metabolic enhancing properties according to claim 1 which include increased mental acuity and positive mental outlook.

10. Metabolic enhancing properties according to claim 1 which include increased energy production and anti-fatigue actions.

11. Octopamine salts according to claim 1 which include inorganic salts of octopamine.

12. Inorganic salts of octopamine according to claim 10 which include but not limited to the hydrochloride, hydrobromide, sulfate, phosphate, nitrate, carbonate, hydroselenate, metasilicate, permanganate, sulfonate, phosponate, etc.

13. Octopamine salts according to claim 1 which include organic salts of octopamine.

14. Organic salts of octopamine according to claim 12 which include carboxylate salts.

15. Carboxylate salts of octopamine according to claim 13 which include but not limited to citrate, malate, formate, tartrate, maleate, fumarate, tartrate, picolinate, nicotinate, pyruvate, succinate, glycinate, glutamate, ascorbate, decanoate, palmitate, oleate, acetate, propionate, etc.

16. Organic salts of octopamine according to claim 12 which include but not limited to organic sulfonic, organic carbonic, organic phosponic, etc. Examples include but are not limited to methanesulfonate, isethionate, toluenesulfonate, ethylcarbonate, methylcarbonate, etc.

17. Bioavailable and environmentally stable octopamine salts according to claim 1 which are administered perorally as tablets, hard or soft gelatin capsules, sachets, or as a powder, including effervescent powder, or liquid.

18. Bioavailable and environmentally stable octopamine salts according to claim 1 which are administered in a daily dosage between 0.1 mg/kg and 50 mg/kg calculated as octopamine base.

19. The dosage according to claim 18 which is most preferable between 5 mg/kg and 15 mg/kg calculated as octopamine base.
